(19) United States
(12) Patent Application Publication
(10) Pub. No.: US 2020/0093789 A1
(43) Pub. Date: Mar. 26, 2020

(54) Cancer Treatment with a CXCL12 Signaling Inhibitor and an Immune Checkpoint Inhibitor

(71) Applicants: The General Hospital Corporation, Boston, MA (US); Aperisys, Inc., Cumming, GA (US)

(72) Inventors: Mark C. Poznansky, Newton Center, MA (US); Stephen J. McCormack, Las Vegas, NV (US); Michael Callahan, Boulder, CO (US); Jeffrey A. Gelfand, Cambridge, MA (US); Huabiao Chen, Winchester, MA (US); Patrick Reeves, Cambridge, MA (US)

(21) Appl. No.: 16/612,109

(22) PCT Filed: May 10, 2018
(86) PCT No.: PCT/US2018/031955

§ 371 Date: Nov. 8, 2019

Related U.S. Application Data
(60) Provisional application No. 62/503,995, filed on May 10, 2017.

(51) Int. Cl. A61K 31/395; A61K 39/395; A61P 35/00; A61K 45/06

(52) U.S. Cl. CPC A61K 31/395; A61K 45/06; A61P 35/00; A61K 39/3955

(57) ABSTRACT

The present invention relates to methods and compositions for treating cancer and enhancing the immune response against cancer cells using the combination of an inhibitor of CXCL12 signaling and an immune checkpoint inhibitor. The invention further relates to compositions and kits of parts comprising an inhibitor of CXCL 12 signaling and an immune checkpoint inhibitor for use in the methods of the invention.

### STATEMENT OF PRIORITY

This application claims the benefit of U.S. Provisional Application Ser. No. 62/503,995, filed May 10, 2017, the entire contents of which are incorporated by reference herein.

### FIELD OF THE INVENTION

The present invention relates to methods and compositions for treating cancer and enhancing the immune response against cancer cells using the combination of an inhibitor of CXCL12 signaling and an immune checkpoint inhibitor.

### BACKGROUND OF THE INVENTION

CXCL12, formerly known as stromal cell-derived factor-1 (SDF-1), is a cytokine that is the natural ligand for CXCR4 receptors as well as CXCR7 receptors. The CXCR4/CXCL12 chemokine receptor/chemokine axis has been shown to be involved in the pathogenesis of a number of hematological and solid malignancies. CXCR4/CXCL12 signaling provides an immunosuppressive barrier that blocks the immune system from attacking the cancer cells through immune evasion mechanisms including the recruitment of myeloid suppressor cells, tumor-infiltrating macrophages, and immunosuppressive regulatory T cells to the tumor and the repulsion of anti-tumor antigen specific CD8⁺ T cells from the intratumoral microenvironment.

Immune checkpoints refer to a number of inhibitory pathways in the immune system that maintain self-tolerance and modulate the duration and amplitude of physiological immune responses. Recently, immune checkpoint inhibitors have been developed for cancer immunotherapy.

There is a need in the art for improved cancer treatments that can overcome the immunosuppressive barrier provided by many cancers and enhance the ability of the immune system to recognize and attack cancer cells.

### SUMMARY OF THE INVENTION

The present invention is based, in part, on the development of methods for overcoming the immunosuppressive barrier produced by many tumors and enhancing the body's immune response against the tumor. In particular, the combination of an inhibitor of CXCL12 signaling to reduce the immunosuppressive barrier and an immune checkpoint inhibitor to stimulate the immune response may have additive or synergistic effects in enhancing treatment efficacy and survival of cancer patients.

Accordingly, one aspect of the invention relates to a method of treating cancer in a subject in need thereof, comprising delivering to the subject an inhibitor of CXCL12 signaling and an immune checkpoint inhibitor, thereby treating the cancer.

Another aspect of the invention relates to a method for enhancing an immune response against a cancer in a subject in need thereof, comprising delivering to the subject an inhibitor of CXCL12 signaling and an immune checkpoint inhibitor, thereby enhancing the immune response against the cancer.

A further aspect of the invention relates to a composition comprising an inhibitor of CXCL12 signaling and an immune checkpoint inhibitor.

An additional aspect of the invention relates to a kit of parts comprising a first container comprising an inhibitor of CXCL12 signaling and a second container comprising an immune checkpoint inhibitor.

Another aspect of the invention relates an inhibitor of CXCL12 signaling and an immune checkpoint inhibitor for use in treating cancer.

A further aspect of the invention relates an inhibitor of CXCL12 signaling and an immune checkpoint inhibitor for use in enhancing an immune response against a cancer.

An additional aspect of the invention relates to the use of an inhibitor of CXCL12 signaling and an immune checkpoint inhibitor in the preparation of a medicament for treating cancer.

Another aspect of the invention relates to the use of an inhibitor of CXCL12 signaling and an immune checkpoint inhibitor in the preparation of a medicament for enhancing an immune response against a cancer.

These and other aspects of the invention are set forth in more detail in the description of the invention below.

### BRIEF DESCRIPTION OF THE DRAWINGS

FIG. 1 shows the AMD3100/checkpoint inhibitor combination study in a murine ovarian cancer model. Tumors were initiated in immunocompetent C57B1/6 mice by intraperitoneal (IP) injection of the syngeneic ID8 murine ovarian cancer cell line. A luciferase-expressing ID8 line was utilized to allow tumor initiation to be confirmed with an IVIS® in vivo imaging system at day 20 post tumor inoculation. Drugs were administered IP, on alternating days beginning 21 days after tumor initiation, for three weeks as indicated.

FIGS. 2A-2B show the survival of mice in the AMD3100/checkpoint inhibitor combination study. FIG. 2A: Survival of animals after tumor inoculation is plotted for all of the different treatment groups. FIG. 2B: Survival of treatment groups receiving AMD3100 at 3mg/kg, αPD-1, and AMD3100 3mg/kg+αPD-1 combination relative to the saline treated control group. Survival differences were assessed by the Log-rank test.

FIGS. 3A-3B show the proportion of intratumoral CD8⁺ T cells after treatment as a percent of all cells in tumors. FIG. 3A: Intratumoral CD8⁺ T cells of all the groups; FIG. 3B: Intratumoral CD8⁺ T cells of treatment groups with 3mg/kg AMD3100, αPD-1, and 3 mg/kg AMD3100+αPD-1 combination relative to the saline treated control group. Statistical differences were analyzed using One-Way ANOVA. *P<0.05, **P<0.01, ***P<0.001 and ****P<0.0001. Data are presented as mean±SEM.

FIGS. 4A-4B show the proportion of intratumoral T_{reg} cells after treatment as a percent of total CD4⁺ T cells in tumors. FIG. 4A: Intratumoral T_{reg} cells of all the groups; FIG. 4B: Intratumoral L_{reg} cells of treatment groups with 3 mg/kg AMD3100, αPD-1, and 3 mg/kg AMD3100+αPD-1 combination relative to the saline treated control group. Statistical differences were analyzed using One-Way ANOVA. *P<0.05, **P<0.01, ***P<0.001 and ****P<0.0001. Data are presented as mean ±SEM.

FIGS. 5A-5B show the ratio of intratumoral CD8⁺ T cells to T_{reg} cells after treatment. FIG. 5A: Intratumoral CD8⁺/T_{reg} ratios of all the groups; FIG. 5B: Intratumoral CD8⁺/T_{reg} ratios of treatment groups with 3 mg/kg AMD3100, αPD-1, and 3 mg/kg AMD3100+αPD-1 combination relative to the saline treated control group. Statistical differences were analyzed using One-Way ANOVA. *P<0.05, **P<0.01,***P<0.001 and ****P<0.0001. Data are presented as mean ±SEM.

FIGS. 6A-6B show the proportion of intratumoral memory CD8⁺ (CD44⁺CD27⁺) T cells after treatment as a percentage of total CD8⁺ T cells in tumors. FIG. 6A: Intratumoral memory CD8⁺ T cells of all the groups; FIG. 6B: Intratumoral memory CD8⁺ T cells of treatment groups with 3 mg/kg AMD3100, αPD-1, and 3 mg/kg AMD3100+αPD-1 combination relative to the saline treated control group. Statistical differences were analyzed using One-Way ANOVA. *P<0.05, **P<0.01, ***P<0.001 and ****P<0.0001. Data are presented as mean±SEM.

FIGS. 7A-7B show the proportion of intratumoral interferon-γ-expressing CD8 T cells after treatment as a percentage of total CD8⁺ T cells in tumors. FIG. 7A: Intratumoral IFNγ-expressing CD8⁺ T cells of all the groups; FIG. 7B: Intratumoral IFNγ-secreting CD8⁺ T cells of treatment groups with 3 mg/kg AMD3100, αPD-1, and 3 mg/kg AMD3100+αPD-1 combination relative to the saline treated control group. Statistical differences were analyzed using One-Way ANOVA. *P<0.05, **P<0.01, ***P <0.001 and ****P<0.0001. Data are presented as mean±SEM.

FIGS. 8A-8B show the proportion of helper T cell phenotypic IL-2⁺CD40L⁺ cells as a percentage of total CD4⁺CD25⁻FoxP3⁺ cells in tumors. FIG. 8A: Intratumoral IL-2⁺CD40L⁺ cells of all the groups; FIG. 8B: Intratumoral IL-2⁺CD40L⁺ cells of treatment groups with 3 mg/kg AMD3100, αPD-1, and 3 mg/kg AMD3100 +αPD-1 combination relative to the saline treated control group. Statistical differences were analyzed using One-Way ANOVA. *P<0.05, **P<0.01, ***P<0.001 and ****P<0.0001. Data are presented as mean±SEM.

FIGS. 9A-9B show the percentage of myeloid-derived suppressor cells (MDSCs) after treatment as a percentage of total cells in tumors. FIG. 9A: Intratumoral MDSCs of all the groups; FIG. 9B: Intratumoral MDSCs of treatment groups with 3 mg/kg AMD3100, αPD-1, and 3 mg/kg AMD3100+αPD-1 combination relative to the saline treated control group. Statistical differences were analyzed using One-Way ANOVA. *P<0.05, **P<0.01, ***P<0.001 and ****P<0.0001. Data are presented as mean±SEM.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof as used with respect to a polypeptide and a receptor, refers to bringing the polypeptide and the receptor in sufficiently close proximity to each other for one to exert a biological effect on the other. In some embodiments, the term contact means binding of the polypeptide to the receptor.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

By the terms “treat,” “treating,” or “treatment of,” it is intended that the severity of the subject's condition is reduced or at least partially improved or modified and that some alleviation, mitigation or decrease in at least one clinical symptom is achieved.

“Antibodies” as used herein include polyclonal, monoclonal, single chain, chimeric, humanized and human antibodies, prepared according to conventional methodology.

“CXCL12 signaling” as used herein refers to the signaling pathways that involve binding of CXCL12 to its receptors, including CXCR4 and CXCR7.

A first aspect of the invention relates to a method of treating cancer in a subject in need thereof, comprising delivering to the subject an inhibitor of CXCL12 signaling and an immune checkpoint inhibitor, thereby treating the cancer.

Another aspect of the invention relates to a method for enhancing an immune response against a cancer in a subject in need thereof, comprising delivering to the subject an inhibitor of CXCL12 signaling and an immune checkpoint inhibitor, thereby enhancing the immune response against the cancer. The term “enhancing an immune response against a cancer,” as used herein, refers to increasing the ability of the immune system to recognize and/or attack cancer cells. Enhancements may include, without limitation, an increase in tumor-specific cytotoxic lymphocytes, CD8⁺ lymphocytes capable of delivering to the tumor Granzyme B, interferon, perform, and/or Fas ligand, and/or a decrease in the co-location of T lymphocyte suppressor or regulatory cells and/or myeloid-derived suppressor cells, or any combination of the above.

The inhibitor of CXCL12 signaling may be any molecule that inhibits the CXCL12/CXCR4 and/or CXCL12/CXCR7 axis. The inhibitor may completely or partially inhibit signaling through the CXCL12/CXCR4 and/or CXCL12/CXCR7 axis when administered to a subject, e.g., providing at least about 30%, 40%, 50%, 60%, 70%, 80%, 90% or more inhibition. Inhibitors may include, without limitation, molecules that inhibit expression of CXCL12 or CXCR4 or CXCR7 (e.g., antisense or siRNA molecules), molecules that specifically bind to CXCL12 or CXCR4 or CXCR7 and inhibit their function (e.g., antibodies or aptamers), molecules that inhibit dimerization of CXCL12 or CXCR4 or CXCR7, and antagonists of CXCR4 or CXCR7. In one embodiment, the inhibitor of CXCL12 signaling is a CXCR4 antagonist. The CXCR4 antagonist can be but is not limited to AMD3100, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, or TN14003, or an antibody that interferes with the dimerization of CXCR4. Additional CXCR4 antagonists are described, for example, in U.S. Patent Pub. No. 2014/0219952 and Debnath et al. (Theranostics, 2013; 3 (1): 47-75), each of which is incorporated herein by reference in its entirety, and include TG-0054 (burixafor), AMD3465, NIBR1816, AMD070, and derivatives thereof In one embodiment, the CXCR4 antagonist is AMD3100 (plerixafor). AMD3100 is described in U.S. Pat. No. 5,583,131, which is incorporated by reference herein in its entirety. In one embodiment, the inhibitor of CXCL12 signaling is a CXCR7 antagonist. The CXCR7 antagonist can be but is not limited to CCX771, CCX754, or an antibody that interferes with the dimerization of CXCR7. In certain embodiments, the inhibitor of CXCL12 signaling is a small molecule, e.g., less than 1500, 1000, 900, 800, 700, 600, or 500 Da. In certain embodiments, the inhibitor of CXCL12 signaling is not an antibody. In certain embodiments, the inhibitor of CXCL12 signaling is not a heparinoid. In certain embodiments, the inhibitor of CXCL12 signaling is not a peptide.

In some embodiments, CXCR4 inhibitors useful with the invention may include, but are not limited to, CXCL12 (SDF-1) mutants, fusion proteins/genes, truncations and/or analogues. Non-limiting examples of CXCL12 mutants for inhibition CXCR4 include SDF-1P2G54 (Yang et al. Nan Fang Yi Ke Da Xue Xue Bao:32 (1):55-60 (2012)), SDF-1α/54/KDEL (Chen et al. J. Interferon Cytokine Res. 35 (1):771-778 (2015)), SDF-1/54 (Ma et al. Biol. Pharm. Bull. 31 (6):1086-1091 (2008)), CXCL12α (Δ8 L29K V39K) (Gschwandtner et al. FEBS Letters 589:2819-2824 (2015)), P2G (SDF-1β mutant) (Yan et al. J. Cell Mol. Med. epub doi: 10.1111/jcmm.13150 (2017), mutants in which the N-terminal 1-8aa residues are replaced with all D-stereoisomers of 1-10aa sequence of vMIP-II: RXP168 (e.g., D-(1-10)-vMIP-II) and RCP222 (D(1-10)-vMIP-II-(9-68)-CXCL12α) (Patrussi et al. Current Medicinal Chemistry 18:497-512 (2011))), and hSDF-154 (Tan et al. Exp. Hematol. 34:1553-1562 (2006)). Non-limiting examples of CXCL12 fusion proteins/genes that may be useful as CXCR4 inhibitors include SDF-1/54-DCN (Ma et al. Biol. Pharm. Bull. 31 (6):1086-1091 (2008)), CXCL12-Ig (Meiron et al. J. Exp Med. 205 (11):2643-2655 (2008)), CXCL122 (Takekoshi et al. Mol. Cancer Ther. 11 (11):2516-2525 (2012)), CXCL12-KDEL (Zhang et al. Biomed. Res. Int. Vol. 2015, Article ID 195828, 9 pages (2015) and Zhang et al. J. Virol. Methods 161:30-37 (2009)), BIGFI-SDF-1 (bombyxin-IGFI chimera) (Sandoval et al. Biochem. Pharmacol. 65:2055-2063 (2003)), and affinity tagged CXCL12 (e.g., CXCL12-HIS, CXCL12-Strep-LT, and CXCL12-HIS-LT (Picciocchi et al. PLoS One 9 (1):e87394 (2014)). Non-limiting examples of CXCL12 truncation variants that may be useful as inhibitors of CXCR4 include CXCL12[29-88] (Richter et al. Stem Cells Dev. 23 (16):1959-1974 (2014)), N-addition mutants (e.g., FNQL-CXCL12, LGGG-CXCL12, LRHQ-CXCL12, LRSQ-CXCL12, MLGI-CXCL12, MRHQ-CXCL12, VPGA-CXCL12, QWVA-CXCL12, QFNI-CXCL12, SQCS-CXCL12, SQSQ-CXCL12, SQLA-CXCL12 (Hanes et al. J. Biol. Chem. 290 (37):22385-22397 (2015))), CXCL12(3-68) (CD26 truncated CXCL12) (Janssens et al. Biochem. Pharmacol. 132:92-101 (2017)) and CXCL12βT (51-72 aa of CXCL12β) and CXCL12α T (51-68 aa of CXCL12α) (Patrussi et al. Current Medicinal Chemistry 18:497-512 (2011)). A non-limiting example of a CXCL12 analogue that may be useful as inhibitors of CXCR4 includes CTCE-9908 (17 amino acid analogue of CXCL12) (Patrussi et al. Curr. Med. Chem. 18:497-512 (2011)).

The immune checkpoint inhibitor may be any molecule that inhibits an immune checkpoint Immune checkpoints are well known in the art and include, without limitation, PD-1, PD-L1, PD-L2, CTLA4, B7-H3, B7-H4, BTLA, IDO, KIR, LAG3, A2AR, TIM-3, and VISTA. In some embodiments, the inhibitor is an antibody against the immune checkpoint protein. In certain embodiments, the immune checkpoint inhibitor is an inhibitor of PD-1 or PD-L1, e.g., an antibody that specifically binds PD-1 or PD-L1. In some embodiments, the immune checkpoint inhibitor is nivolumab, pembrolizumab, ipilimumab, durvalumab, or atezolizumab. In one embodiment, the immune checkpoint inhibitor is nivolumab.

Inhibitors of CXCL12 signaling and immune checkpoint inhibitors are known in the art, for example, see U.S. Publication Nos. 2016/0235779, 2016/0304607, 2015/0352208, and 2015/0216843 and International Publication Nos. WO 2017/019767, WO 2017/009842, and WO 2016/201425.

Non-limiting examples of immune checkpoint inhibitors useful with this invention are provided in Table 1.

TABLE 1
Immune checkpoint inhibitors
10A7
10C7
10F.9G2 (BioXcell)
11159-H03H (Sino Biological Inc.)
11159-H08H (Sino Biological Inc.)
12A4
1D8
1F4 (Roche)
1-methyl tryptophan (1-MT)
1-methyl-tryptophan (1MTrp)
20H4.9-IgG1
20H4.9-IgG4
244C8
29E.2A3
388D4
3E1
3H3
4E9
53A2
9H10 (Millipore)
A110
A9H12
AB134090 (ABCAM ®)
Abciximab
ABR002
Adalimumab
AGEN-1884
AGEN-2034
AK-103
AK-104
AK-105
AK-106
AK-112
Alemtuzumab
ALN-PDL
AM-0001
AMG 228
AMG-557 (Amgen)
AMP-224 (GlaxoSmithKline/Amplimmune)
AMP-514 (Amplimmune/AZ)
ANA011
ANB011
anti-CTLA-4/anti-PD-1 bispecific humanized
antibody (Akeso Biopharma)
anti-PD1 oncolytic monoclonal antibody (Transgene SA)
anti-PD-1NEGF-A DARPins (Molecular Partners)
AP-105
AP-106
Arelumab (Merck Serono)
ASP8374
Atezolizumab (aka MPDL3280A, RG7446, R05541267)
(Tecentriq ®) (Hoffman-La Roche)
AUNP 12 (Aurigene and Pierre Fabre)
AUR-012
Avelumab (Bavencio ®) (Merck) (EMD Serono)
B7-H1 vaccine (State Key Laboratory of Cancer
Biology/Fourth Military Medical University)
Basiliximab
Bavituximab
BCD-100
Bcvacizumab (Avastin ®)
BGB-108
BGB-A317
BI-754091
BITES ®
BKT140
Blinatumomab
BMS-469492
BMS-469497
BMS-554271
BMS-663031
BMS-663513
BMS-936558/MDX-1106/ONO-4538 (Medarex, Inc.)
BMS-936559 (Bristol-Myers Squibb)
BMS-986016 (Bristol-Myers Squibb)
BMS-986153
BMS-986156
BN13 (Abeam)
Brentuximab vedotin
CA-170
CBT-501
CBT-502
CC-90002
CDX-1127 (Celldex Therapeutics)
Cetuximab (Erbitux ®)
Codrituzumab
CP-870,893 (Genentech)
CVT-6883
CX-072
Dacetuzumab
Daclizumab
Daratumumab
DARTs ®
dextro-1-methyl tryptophan (D-1MT)
dual targeting anti-PD-1/LAG-3 mAbs (Tesaro)
dual targeting anti-PD-1/TIM-3 mAbs (Tesaro)
DUOBODIES ®
Durvalumab/MEDI-4736 (Anti-B7-H1)
(Imfinzi ™) (Med immune)
eBioC9B7W (C9B7W) (eBioscience)
EH12
Emactuzumab
Enoblituzumab (MGA271)
ENUM-244C8
Etanercept
F001287 (Bristol Meyers Squibb)
FAZ-053
FPT-155
FS-118
Galiximab (Biogen Idec)
Galunisertib
GB-226
Gemtuzumab ozogamicin
GLS-010
GNS-1480
GX-D1
GX-P2
HVEM
IBI-308
Ibritumomab tiuxetan
IgG CD134 mAb
IMP-321 (Immutep S.A.)
IMP-731
INCB023843
INCB024360 (Incyte)
Indoximod (D-1-methyl tryptophan
(D-1-MT)) (NewLink Genetics)
INDUS-903
Infliximab
INSIX RA
IO-102
IO-103
IPH2101 (also called 1-7F9) (Innate Pharma)
IPH2201
Ipilimumab/MDX-010/BMS-734016
(Yervoy ™) (Bristol-Myers Squibb)
Istradelylline
J43 (BioXcell)
Jienuo mAb (Genor Biopharma)
JNJ-63723283
JS001
JTX-2011 (Jounce Therapeutics)
JTX-4014
KAHR-102
KD-033
KN044
KY-1003
L-1-methyl tryptophan (L-1-MT)
LAG3
Lambrolizumab (MK-3475)
Lirilumab (aka BMS-986015, IPH2102)
(Innate Pharma) (Bristol-Myers Squibb)
LS-B2237 (LifeSpan Biosciences)
Lucatumumab
LY-3300054
M-7824
MAI-12205 (Thermo Scientific Pierce)
MAI-35914 (Thermo Scientific Pierce)
MBG219
MBG220
MBG227
MCLA-134
MCLA-145
MDPL3280A (Genentech/Roche)
MDX-010 (Ipilimumab)
MDX-1105 (Medarex, Inc./Bristol Myers Squibb)
MDX-400
MED16383 (rOX40L)
MEDI-0562
MEDI-0570 (MedImmune, LLC)
MEDI-0608 (formerly AMP-514)
MEDI-0680
MEDI-4736 (Medimmune/AstraZeneca)
MEDI-6383
MEDI-6469 (MedImmune/AZ)
Merck 3475/MK-3475/SCH-900475 (Merck)
MGA-012
MGA271 (Macrogenics)
MGD-013
Mivolumab
MK-4166
MNRP1685A
Mogamulizumab (Kyowa Hakko Kirin)
MOXR0916 (Genetech)
MPDL3280A (aka RG7446) (Roche)
MRS-1706
MRS-1754
MSB-0010718C (aka A09-246-2) (Merck Serono)
MSB-2311
muDX400
Nimotuzumab
Nivolumab (aka BMS-936558, MDX-1106,
ONO-4538) (Opdivo ®) (Bristol-Myers Squibb)
NLG-919 (NewLink Genetics)
NSC-721782 (also known as 1-methyl-D-tryptophan)
Obinutuzumab
Ocaratuzumab
Ocrelizumab
Ofatumumab
Palivizumab
Panitumumab
PAS-23967 (Thermo Scientific Pierce)
PAS-26465 (Thermo Scientific Pierce)
PAS-29572 (Thermo Scientific Pierce)
PD-1 based bispecific antibody (Beijing
Hanmi Pharmaceutical)
PD-1 inhibitor peptide (Aurigene)
PD-1 modified TILs (Sangamo Therapeutics)
PD-1/CTLA-4 bispecific antibody (MacroGenics)
PD-1/Ox40 targeting bispecific antibody
(Immune Pharmaceuticals)
PD1/PDL1 inhibitor vaccine (THERAVECTYS)
PD-L1 targeting CAR-TNK-based immunotherapy
(TNK Therapeutics/NantKwest)
PD-L1/BCMA bispecific antibody (Immune
Pharmaceuticals)
PD-L1-TGF-beta therapy
PDR-001
Pelareorep
Pembrolizumab (aka MK-3475, SCH 900475, or
Lambrolizumab) (Keytruda ®) (Merck)
PF-04518600 (Pfizer)
PF-05082566 (anti-4-1BB, PF-2566) (Pfizer)
PF-06801591
Pidilizumab (aka CT-011, MDV9300) (CureTech, Ltd.)
PRO304397
Probody targeting PD-1 (CytomX)
PRS-332
PSB-0788
PSB-1115
PSB-603
PSI-001
Racotumomab (formerly known as 1E10)
Recombinant humanized anti-PD-1 mAb
(Bio-Thera Solutions)
Recombinant humanized anti-PD1 mAb
(Shanghai Junshi Biosciences)
REGN-1979
REGN-2810
Resminostat
RG-7446
RG-7888
rHIgM12B7
rhTL-15
Rituximab (Mabthera ®)
RMP1-14 (BioXCell)
SCH-442,416
SCH-58261
SHR-1210
SHR-1316
siRNA loaded dendritic cell vaccine
(Alnylam Pharmaceuticals)
SN-PD07
SN-PDL01
SP142
SSI-361
STI-1014
STI-1110
STI-A1010
STI-A1011
STI-A1012
STI-A1014
STI-A1110
TandAbs ®
Ticilimumab
Tositumomab
Trastuzumab
Tremelimumab (aka CP-675,206, CP-675,
CP-675206, and Ticilimumab) (Pfizer)
TRU-015
TRX518 (GITR, Inc.)
Tryptophan
Tryptophan mimetic
TSR-022
TSR-042
TX-2274
UC10-4F10-11 (BioXCell)
Ulocuplumab
Urelumab (BMS-663513) (Bristol-
Myers Squibb)
V domain-containing Ig suppressor of T-cell
activation (VISTA)
Varlilumab (CelIDex Therapeutics)
Veltuzumab
WBP-3155
XCE853
XmAb-20717
XmAb-5592 (Xencor)
XmAbs ®
YW243.55.S70
ZM-241,365
ZM-241,385

The inhibitor of CXCL12 signaling and the immune checkpoint inhibitor may be delivered to the subject in any manner or pattern that is effective. In some embodiments, the inhibitor of CXCL12 signaling and the immune checkpoint inhibitor are delivered to the subject in the same composition. In other embodiments, the inhibitor of CXCL12 signaling and the immune checkpoint inhibitor are delivered to the subject in separate compositions. The two agents may be delivered to the subject simultaneously. The two agents may be delivered to the subject sequentially and the sequence may be repeated as necessary, e.g., repeated 1, 2, 3, 4, 5, 6, 7, 8, 9, or 10 time or more. In some embodiments, either the inhibitor of CXCL12 signaling or the immune checkpoint inhibitor may be administered alone for a period of time, following by a combination of the two agents. For example a subjected may be administered an inhibitor of CXCL12 signaling first (e.g., to allow infiltration of immune cells into the tumor), followed by administration of the combination. In some embodiments, a delivery regimen of the invention may be carried out, stopped for a period of time, then resumed. For example, the delivery may be halted for a period of time so that other treatments or surgeries may be applied. The delivery may then be resumed. This pattern may be repeated as necessary to improve the overall efficacy of the treatment. The break in delivery of the method of the invention may enable newly infiltrated immune cells to combat the tumor for a longer period of time. The method may be resumed when the efficacy benefit is anticipated or measured to be reduced.

In certain embodiments, the two agents may be delivered in the same pattern and/or schedule. In other embodiments, the two agents may be delivered in a different pattern and/or schedule. In some embodiments, the immune checkpoint inhibitor may be administered to the subject for a sufficient amount of time to stimulate the immune system and then stopped. For example, the immune checkpoint inhibitor may be administered for just a few doses, e.g., 10 doses or less, e.g., 10, 9, 8, 7, 6, 5, 4, 3, 2, or 1 dose, in a periodic fashion, e.g., once every week, 2 weeks, 3 weeks, 4 weeks, 5 weeks, 6 weeks, or more. In some embodiments, the inhibitor of CXCL12 signaling may be administered for a longer period of time than the immune checkpoint inhibitor, e.g., until the cancer has been successfully treated. The inhibitor of CXCL12 signaling also may be administered more frequently than the immune checkpoint inhibitor, e.g., once every 3 hours, 4 hours, 6 hours, 12 hours, day, 2 days, 3 days, 4 days, 5, days, 6, days, week, or more.

The cancer to be treated may be any cancer for which the methods of the invention are effective. In some embodiments, the cancer is a solid tumor. In other embodiments, the cancer is a not a solid tumor, e.g., a leukemia. In some embodiments, the cancer is one that expresses CXCL12 and/or CXCR4 and/or CXCR7. Cancers that are known to express CXCL12 include, without limitation, cancers of the bone marrow, cerebral cortex, parathyroid gland, thyroid gland, nasopharynx, lung, gallbladder, small intestine, kidney, testis, seminal vesicle, placenta, skin, hippocampus, caudate, cerebellum, lymph node, tonsil, spleen, heart muscle, bronchus, pancreas, duodenum, oral mucosa, urinary bladder, vagina, uterine cervix, endometrium, soft tissue, and colon. Cancers that are known to express CXCR4 include, without limitation, cancers of the bone marrow, tonsil, lymph node, appendix, spleen, adrenal gland, gallbladder, fallopian tube, placenta, endometrium, small intestine, uterine cervix, rectum, stomach, prostate, lung, breast, smooth muscle, esophagus, adipose tissue, colon, thyroid gland, liver, kidney, seminal vesicle, ovary, salivary gland, duodenum, cerebral cortex, heart muscle, parathyroid gland, epididymis, testis, skin, pancreas, and skeletal muscle. Cancers that are known to express CXCR7 include, without limitation, cancers of the thyroid gland, appendix, pancreas, kidney, fallopian tube, placenta, lymph node, tonsil, smooth muscle, skeletal muscle, heart muscle, bronchus, lung, liver, gallbladder, esophagus, salivary gland, rectum, stomach, duodenum, small intestine, colon, urinary bladder, testis, epididymis, breast, endometrium, adipose tissue, soft tissue, and skin.

Cancers or tumors that can be treated by the compounds and methods described herein include any of the cancers listed above and include, but are not limited to: biliary tract cancer; brain cancer, including glioblastomas and medulloblastomas; breast cancer; cervical cancer, including carcinoma in situ, squamous cell carcinoma, adenocarcinoma, adenosquamous carcinoma, small cell carcinoma, neuroendocrine tumor, villoglandular adenocarcinoma, and glassy cell carcinoma; endocrine tumors, including insulinoma; choriocarcinoma; colon cancer; endometrial cancer; esophageal cancer, gastric cancer; head and neck cancers; hematological neoplasms, including acute lymphocytic and myelogenous leukemia; multiple myeloma; AIDS associated leukemias and adult T-cell leukemia lymphoma; intraepithelial neoplasms, including Bowen's disease and Paget's disease; liver cancer (hepatocarcinoma); lung cancers; lymphomas, including Hodgkin's disease and lymphocytic lymphomas; mesothelioma, neuroblastomas; oral cancer, including squamous cell carcinoma; ovarian cancer, including those arising from epithelial cells, stromal cells, germ cells and mesenchymal cells; pancreas cancer; peritoneal cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, Merkel cell carcinoma, basocellular cancer and squamous cell cancer; testicular cancer, including germinal tumors (seminoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilms tumor. In some embodiments, the cancer is a human papilloma virus (HPV)-positive cancer, e.g., cervical cancer or head and neck cancer. In certain embodiments, the cancer is a cancer that is unresponsive to PD-1 blockade. Precancerous lesions may also be treated by the methods of the present invention, including, without limitation, cervical intraepithelial neoplasia 1, 2, and 3.

In some embodiments, the cancer is ovarian cancer, fallopian tube cancer, cervical cancer, vaginal cancer, rectal cancer, pancreatic cancer, cholangiocarcinoma, peritoneal cancer, mesothelioma, non-small cell lung cancer, kidney cancer, bladder cancer, Hodgkin lymphoma, or head and neck cancer. In some embodiments, the cancer is ovarian cancer.

The methods of the invention may further comprise delivering to the subject one or more anti-cancer agents or treatments, e.g., a chemotherapeutic agent and/or a radiotherapeutic agent and/or an immunotherapeutic agent. The methods of the invention may further comprise surgery to remove some or all of the tumor and/or post-surgery disease reduction. The methods of the invention may further comprise adjunctive treatments that enhance the therapeutic effect, e.g., administering an HPV vaccine to an HPV-positive cancer.

The chemotherapeutic agent may be any agent having a therapeutic effect on one or more types of cancer. Many chemotherapeutic agents are currently known in the art. Types of chemotherapy drugs include, by way of non-limiting example, alkylating agents, antimetabolites, anti-tumor antibiotics, topoisomerase inhibitors, mitotic inhibitors, corticosteroids, and the like.

Non-limiting examples of chemotherapeutic drugs include: nitrogen mustards, such as mechlorethamine (nitrogen mustard), chlorambucil, cyclophosphamide (Cytoxan®), ifosfamide, and melphalan); nitrosoureas, such as streptozocin, carmustine (BCNU), and lomustine; alkyl sulfonates, such as busulfan; triazines, such as dacarbazine (DTIC) and temozolomide(Temodar®); ethylenimines, such as thiotepa and altretamine (hexamethylmelamine); platinum drugs, such as cisplatin, carboplatin, and oxalaplatin; 5-fluorouracil (5-FU); 6-mercaptopurine (6-MP); capecitabine (Xeloda®); cytarabine (Ara-C®); floxuridine; fludarabine; gemcitabine (Gemzar®); hydroxyurea; methotrexate; pemetrexed (Alimta®); anthracyclines, such as daunorubicin, doxorubicin (Adriamycin®), epirubicin, idarubicin; actinomycin-D; bleomycin; mitomycin-C; mitoxantrone; topotecan; irinotecan (CPT-11); etoposide (VP-16); teniposide; mitoxantrone; taxanes: paclitaxel (Taxol®) and docetaxel (Taxotere®); epothilones: ixabepilone (Ixempra®); vinca alkaloids: vinblastine (Velban®), vincristine (Oncovin®), and vinorelbine (Navelbine®); estramustine (Emcyt®); prednisone; methylprednisolone (Solumedrol®); dexamethasone (Decadron®); L-asparaginase; and bortezomib (Velcade®).

Doses and administration protocols for chemotherapeutic drugs are well-known in the art. The skilled clinician can readily determine the proper dosing regimen to be used, based on factors including the chemotherapeutic agent(s) administered, type of cancer being treated, stage of the cancer, age and condition of the patient, patient size, location of the tumor, and the like. In some embodiments, chemotherapeutic agents may be administered at doses and schedules known in the art to be effective. In some embodiments, when combined with the methods of the present invention, chemotherapeutic agents may be administered at lower doses and/or with less frequency than typically used.

The radiotherapeutic agent may be any such agent having a therapeutic effect on one or more types of cancer. Many radiotherapeutic agents are currently known in the art. Types of radiotherapeutic drugs include, by way of non-limiting example, X-rays, gamma rays, and charged particles. In one embodiment, the radiotherapeutic agent is delivered by a machine outside of the body (external-beam radiation therapy). In one embodiment, the radiotherapeutic agent is placed in the body near the tumor/cancer cells (brachytherapy) or is a systemic radiation therapy.

External-beam radiation therapy may be administered by any means. Exemplary, non-limiting types of external-beam radiation therapy include linear accelerator-administered radiation therapy, 3-dimensional conformal radiation therapy (3D-CRT), intensity-modulated radiation therapy (IMRT), image-guided radiation therapy (IGRT), tomotherapy, stereotactic radiosurgery, photon therapy, stereotactic body radiation therapy, proton beam therapy, and electron beam therapy.

Internal radiation therapy (brachytherapy) may be by any technique or agent. Exemplary, non-limiting types of internal radiation therapy include any radioactive agents that can be placed proximal to or within the tumor, such as radium-226 (Ra-226), cobalt-60 (Co-60), cesium-137 (Cs-137), cesium-131, iridium-192 (Ir-192), gold-198 (Au-198), iodine-125 (1-125), palladium-103, yttrium-90, etc. Such agents may be administered by seeds, needles, or any other route of administration, and may be temporary or permanent.

Systemic radiation therapy may be by any technique or agent. Exemplary, non-limiting types of systemic radiation therapy include radioactive iodine, ibritumomab tiuxetan (Zevalin®), tositumomab and iodine-131 tositumomab (Bexxar®), samarium-153-lexidronam (Quadramet®), strontium-89 chloride (Metastron®), metaiodobenzylguanidine, lutetium-177, yttrium-90, strontium-89, and the like.

In one embodiment, a radiosensitizing agent is also administered to the patient. Radiosensitizing agents increase the damaging effect of radiation on cancer cells.

Doses and administration protocols for radiotherapy agents are well-known in the art. The skilled clinician can readily determine the proper dosing regimen to be used, based on factors including the agent(s) administered, type of cancer being treated, stage of the cancer, location of the tumor, age and condition of the patient, patient size, and the like. In some embodiments, radiotherapeutic agents may be administered at doses and schedules known in the art to be effective. In some embodiments, when combined with the methods of the present invention, radiotherapeutic agents may be administered at lower doses and/or with less frequency than typically used.

Immunotherapeutic agents include natural killer cells, NK-92 cells, T cells, and antibodies.

Natural killer (NK) cells are a class of lymphocytes that typically comprise approximately 10% of the lymphocytes in a human. NK cells provide an innate cellular immune response against tumor and infected (target) cells. NK cells, which are characterized as having a CD⁻/CD56⁺ phenotype, display a variety of activating and inhibitory cell surface receptors. NK cell inhibitory receptors predominantly engage with major histocompatibility complex class I (“MHC-I”) proteins on the surface of a normal cell to prevent NK cell activation. The MHC-I molecules define cells as “belonging” to a particular individual. It is thought that NK cells can be activated only by cells on which these “self MHC-I molecules” are missing or defective, such as is often the case for tumor or virus-infected cells.

NK cells are triggered to exert a cytotoxic effect directly against a target cell upon binding or ligation of an activating NK cell receptor to the corresponding ligand on the target cell. The cytotoxic effect is mediated by secretion of a variety of cytokines by the NK cells, which in turn stimulate and recruit other immune system agents to act against the target. Activated NK cells also lyse target cells via the secretion of the enzymes perform and granzyme, stimulation of apoptosis-initiating receptors, and other mechanisms.

NK cells have been evaluated as an immunotherapeutic agent in the treatment of certain cancers. NK cells used for this purpose may be autologous or non-autologous (i.e., from a donor).

In one embodiment, the NK cells used in the compositions and methods herein are autologous NK cells. In one embodiment, the NK cells used in the compositions and methods herein are non-autologous NK cells.

In one embodiment, the NK cells used in the compositions and methods herein are modified NK cells. NK cells can be modified by insertion of genes or RNA into the cells such that the cells express one or more proteins that are not expressed by wild type NK cells. In one embodiment, the NK cells are modified to express a chimeric antigen receptor (CAR). In a preferred embodiment, the CAR is specific for the cancer being targeted by the method or composition.

Non-limiting examples of modified NK cells can be found, for example, in Glienke, et al. 2015, Advantages and applications of CAR-expressing natural killer cells, Frontiers in Pharmacol. 6, article 21; PCT Patent Pub. Nos. WO 2013154760 and WO 2014055668; each of which is incorporated herein by reference in its entirety.

The NK-92 cell line was discovered in the blood of a subject suffering from a non-Hodgkin's lymphoma. NK-92 cells lack the major inhibitory receptors that are displayed by normal NK cells, but retain a majority of the activating receptors. NK-92 cells are cytotoxic to a significantly broader spectrum of tumor and infected cell types than are NK cells and often exhibit higher levels of cytotoxicity toward these targets. NK-92 cells do not, however, attack normal cells, nor do they elicit an immune rejection response. In addition, NK-92 cells can be readily and stably grown and maintained in continuous cell culture and, thus, can be prepared in large quantities under c-GMP compliant quality control. This combination of characteristics has resulted in NK-92 being entered into presently on-going clinical trials for the treatment of multiple types of cancers.

NK-92 cells used in the compositions and methods described herein may be wild type (i.e., unmodified) NK-92 cells or modified NK-92 cells. NK-92 cells can be modified by insertion of genes or RNA into the cells such that the cells express one or more proteins that are not expressed by wild type NK-92 cells. In one embodiment, NK-92 cells are modified to express a chimeric antigen receptor (CAR) on the cell surface. In one embodiment, the CAR is specific for the cancer being targeted by the method or composition. In one embodiment, NK-92 cells are modified to express an Fc receptor on the cell surface. In one embodiment, the NK-92 cell expressing the Fc receptor can mediate antibody-dependent cell-mediated cytotoxicity (ADCC). In one embodiment, the Fc receptor is CD 16. In one embodiment, NK-92 cells are modified to express a cytokine (e.g., IL-2).

In one embodiment, the modified NK-92 cell is administered in combination with an antibody specific for the cancer to be treated. In one embodiment, the modified NK-92 cell administered in combination with the antibody is competent to mediate ADCC.

Non-limiting examples of modified NK-92 cells are described, for example, in U.S. Pat. Nos. 7,618,817 and 8,034,332; and U.S. Patent Pub. Nos. 2002/0068044 and 2008/0247990, each of which is incorporated herein by reference in its entirety. Non-limiting examples of CAR-modified NK-92 cells can be found, for example, in Glienke, et al. 2015, Advantages and applications of CAR-expressing natural killer cells, Frontiers in Pharmacol. 6, article 21; which is incorporated herein by reference in its entirety.

T cells are lymphocytes having T-cell receptor in the cell surface. T cells play a central role in cell-mediated immunity by tailoring the body's immune response to specific pathogens. T cells, especially modified T cells, have shown promise in reducing or eliminating tumors in clinical trials. Generally, such T cells are modified and/or undergo adoptive cell transfer (ACT). ACT and variants thereof are well known in the art. See, for example, U.S. Pat. Nos. 8,383,099 and 8,034,334, which are incorporated herein by reference in their entireties.

U.S. Patent App. Pub. Nos. 2014/0065096 and 2012/0321666, incorporated herein by reference in their entireties, describe methods and compositions for T cell or NK cell treatment of cancer. T cells can be activated and expanded generally using methods as described, for example, in U.S. Pat. Nos. 6,352,694; 6,534,055; 6,905,680; 6,692,964; 5,858,358; 6,887,466; 6,905,681; 7, 144,575; 7,067,318; 7, 172,869; 7,232,566; 7, 175,843; 5,883,223; 6,905,874; 6,797,514; 6,867,041; and U.S. Patent Application Publication No. 2006/0121005, each of which is incorporated herein by reference in its entirety.

In one embodiment, the T cells used in the compositions and methods herein are autologous T cells (i.e., derived from the patient). In one embodiment, the T cells used in the compositions and methods herein are non-autologous (heterologous; e.g., from a donor or cell line) T cells. In one embodiment, the T cell is a cell line derived from T cell(s) or cancerous/transformed T cell(s).

In one embodiment, the T cell used in the methods and compositions described herein is a modified T cell. In one embodiment, the T cell is modified to express a CAR on the surface of the T cell. In one embodiment, the CAR is specific for the cancer being targeted by the method or composition. In one embodiment, the T cell is modified to express a cell surface protein or cytokine. Exemplary, non-limiting examples of modified T cells are described in U.S. Pat. No. 8,906,682; PCT Patent Pub. Nos. WO 2013154760 and WO 2014055668; each of which is incorporated herein by reference in its entirety.

In one embodiment, the T cell is a T cell line. Exemplary T cell lines include T-ALL cell lines, as described in U.S. Pat. No. 5,272,082, which is incorporated herein by reference in its entirety.

Immunotherapy also refers to treatment with anti-tumor antibodies. That is, antibodies specific for a particular type of cancer (e.g., a cell surface protein expressed by the target cancer cells) can be administered to a patient having cancer. The antibodies may be monoclonal antibodies, polyclonal antibodies, chimeric antibodies, antibody fragments, human antibodies, humanized antibodies, or non-human antibodies (e.g., murine, goat, primate, etc.). The therapeutic antibody may be specific for any tumor-specific or tumor-associated antigen. See, e.g., Scott et al., Cancer Immunity 2012, 12: 14, which is incorporated herein by reference in its entirety.

In one embodiment, the immunotherapy agent is an anti-cancer antibody. Non-limiting examples include trastuzumab (Herceptin®), bevacizumab (Avastin®), cetuximab (Erbitux®), panitumumab (Vectibix®), ipilimumab (Yervoy®), rituximab (Rituxan®), alemtuzumab (Campath®), ofatumumab (Arzerra®), gemtuzumab ozogamicin (Mylotarg®), brentuximab vedotin (Adcetris®), ⁹⁰Y-ibritumomab tiuxetan (Zevalin®), and ¹³¹I-tositumomab (Bexxar®).

The immunotherapeutic agent may be an anti-cancer vaccine (also called cancer vaccine). Anti-cancer vaccines are vaccines that either treat existing cancer or prevent development of a cancer by stimulating an immune reaction to kill the cancer cells. In one embodiment, the anti-cancer vaccine treats existing cancer.

The anti-cancer vaccine may be any such vaccine having a therapeutic effect on one or more types of cancer. Many anti-cancer vaccines are currently known in the art. Such vaccines include, without limitation, dasiprotimut-T, Sipuleucel-T, talimogene laherparepvec, HSPPC-96 complex (Vitespen), L-BLP25, gp1OO melanoma vaccine, and any other vaccine that stimulates an immune response to cancer cells when administered to a patient. The anti-cancer vaccine may be an engineered molecule that targets cancer cells and delivers an immunostimulatory agent. For example, the vaccine may be a fusion protein comprising an antigen targeting portion (e.g., an antibody such as a scFv) and an immunostimulatory portion(e.g., a stress protein such as a heat shock protein). See, for example, U.S. Pat. Nos. 7,749,501 and 8,143,387.

Doses and administration protocols for immunotherapeutic agents are well-known in the art. The skilled clinician can readily determine the proper dosing regimen to be used, based on factors including the agent(s) administered, type of cancer being treated, stage of the cancer, location of the tumor, age and condition of the patient, patient size, and the like. In some embodiments, immunotherapeutic agents may be administered at doses and schedules known in the art to be effective. In some embodiments, when combined with the methods of the present invention, immunotherapeutic agents may be administered at lower doses and/or with less frequency than typically used.

In one embodiment, the inhibitors of the invention are administered directly to a subject. Generally, the inhibitors of the invention will be suspended in a pharmaceutically-acceptable carrier (e.g., physiological saline) and administered orally or by intravenous infusion, or administered subcutaneously, intramuscularly, intrathecally, intraperitoneally, intrarectally, intravaginally, intranasally, intragastrically, intratracheally, or intrapulmonarily. In another embodiment, the intratracheal or intrapulmonary delivery can be accomplished using a standard nebulizer, jet nebulizer, wire mesh nebulizer, dry powder inhaler, or metered dose inhaler. They can be delivered directly to the site of the disease or disorder, such as ovaries, lungs, kidney, or intestines or directly into a tumor.

In one embodiment, the inhibitors are administered proximal to (e.g., near or within the same body cavity as) the tumor, e.g., into the peritoneal or pleural cavity, or topically, e.g., to the skin or a mucosal surface, e.g., to the cervix. In one embodiment, the inhibitors are administered directly into the tumor or into a blood vessel feeding the tumor. In one embodiment, the inhibitors are administered systemically. In a further embodiment, the inhibitors are administered by microcatheter, or an implanted device, or an implanted dosage form.

In one embodiment, the inhibitors are administered in a continuous manner for a defined period. In another embodiment, the inhibitors are administered in a pulsatile manner. For example, the inhibitors may be administered intermittently over a period of time. The agents may be administered in the same or different patterns and for the same or different lengths of time.

The dosage required depends on the choice of the route of administration; the nature of the formulation; the nature of the patient's illness; the subject's size, weight, surface area, age, and sex; other drugs being administered; and the judgment of the attending physician. Wide variations in the needed dosage are to be expected in view of the variety of molecules available and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2-, 3-, 4-, 6-, 8-, 10-, 20-, 50-, 100-, 150-, or more fold). Encapsulation of the inhibitors in a suitable delivery vehicle (e.g., polymeric microparticles, slow release polymeric gels, or implantable devices) may increase the efficiency of delivery, particularly for oral delivery or delivery into or nearby the location of a tumor, e.g., the peritoneal or pleural cavity.

Generally, the dose of each of the inhibitors of the present invention is from about 0.01 mg/kg body weight per day to about 100 mg/kg per day, e.g., about 0.1 mg/kg body weight per day to about 50 mg/kg per day, inclusive of all values and ranges therebetween, including endpoints. In one embodiment, the dose is from about 0.1 mg/kg to about 50 mg/kg per day. In one embodiment, the dose is from about 0.1 mg/kg to about 40 mg/kg per day. In one embodiment, the dose is from about 0.1 mg/kg to about 30 mg/kg per day. In one embodiment, the dose is from about 0.1 mg/kg to about 20 mg/kg per day. In one embodiment, the dose does not exceed about 50 mg per day.

In one embodiment, the dose is from about 0.1 mg/kg per week to about 350 mg/kg per week, inclusive of all values and ranges therebetween, including endpoints. In one embodiment, the dose is about 0.1 mg/kg per week. In one embodiment, the dose is about 1 mg/kg per week. In one embodiment, the dose is about 2 mg/kg per week. In one embodiment, the dose is about 5 mg/kg per week. In one embodiment, the dose is about 10 mg/kg per week. In one embodiment, the dose is about 20 mg/kg per week. In one embodiment, the dose is about 30 mg/kg per week. In one embodiment, the dose is about 40 mg/kg per week. In one embodiment, the dose is about 50 mg/kg per week. In one embodiment, the dose is about 60 mg/kg per week. In one embodiment, the dose is about 70 mg/kg per week. In one embodiment, the dose is about 80 mg/kg per week. In one embodiment, the dose is about 90 mg/kg per week. In one embodiment, the dose is about 100 mg/kg per week. In one embodiment, the dose is about 110 mg/kg per week. In one embodiment, the dose is about 120 mg/kg per week. In one embodiment, the dose is about 130 mg/kg per week. In one embodiment, the dose is about 140 mg/kg per week. In one embodiment, the dose is about 150 mg/kg per week. In one embodiment, the dose is about 160 mg/kg per week. In one embodiment, the dose is about 170 mg/kg per week. In one embodiment, the dose is about 180 mg/kg per week. In one embodiment, the dose is about 190 mg/kg per week. In one embodiment, the dose is about 200 mg/kg per week. In one embodiment, the dose is about 210 mg/kg per week. In one embodiment, the dose about 220 mg/kg per week. In one embodiment, the dose is about 230 mg/kg per week. In one embodiment, the dose is about 240 mg/kg per week. In one embodiment, the dose is about 250 mg/kg per week. In one embodiment, the dose is about 260 mg/kg per week. In one embodiment, the dose is about 270 mg/kg per week. In one embodiment, the dose is about 280 mg/kg per week. In one embodiment, the dose is about 290 mg/kg per week. In one embodiment, the dose is about 300 mg/kg per week. In one embodiment, the dose is about 310 mg/kg per week. In one embodiment, the dose is about 320 mg/kg per week. In one embodiment, the dose is about 330 mg/kg per week. In one embodiment, the dose is about 340 mg/kg per week. In one embodiment, the dose is about 350 mg/kg per week.

In one aspect of the invention, administration of one or both of the inhibitors is pulsatile. In one embodiment, an amount of one or both of the inhibitors is administered every 1 hour to every 24 hours, for example every 1 hour, 2 hours, 3 hours, 4 hours, 5 hours, 6 hours, 7 hours, 8 hours, 9 hours, 10 hours, 11 hours, 12 hours, 13 hours, 14 hours 15 hours, 16 hours, 17 hours, 18 hours, 19 hours, 20 hours, 21 hours, 22 hours, 23 hours, or 24 hours. In one embodiment, an amount of one or both of the inhibitors is administered every 1 day, 2 days, 3 days, 4 days, 5 days, 6 days; 7 days, 8 days, 9 days, or 10 days.

In certain embodiments, the administration of the inhibitors may be of indefinite duration, to be determined by the managing physician, and only terminated when the disease is judged to be either cured or in remission. In some embodiments, a delivery regimen of the invention may be carried out, stopped for a period of time (e.g., to carry out other treatments or surgeries), then resumed.

In one embodiment, administration of the inhibitor of CXCL12 signaling and the immune checkpoint inhibitor is alternated. In one embodiment, administration of the inhibitor of CXCL12 signaling and the immune checkpoint inhibitor is alternated until the condition of the patient improves. Improvement includes, without limitation, reduction in size of the tumor and/or metastases thereof, elimination of the tumor and/or metastases thereof, remission of the cancer, and/or attenuation of at least one symptom of the cancer.

According to certain embodiments, the inhibitors can be targeted to specific cells or tissues in vivo. Targeting delivery vehicles, including liposomes and targeted systems are known in the art. For example, a liposome or particle can be directed to a particular target cell or tissue, e.g., a cancer cell or tumor, by using a targeting agent, such as an antibody, soluble receptor or ligand, incorporated with the liposome or particle, to target a particular cell or tissue to which the targeting molecule can bind. Targeting liposomes are described, for example, in Ho et al., Biochemistry 25:5500 (1986); Ho et al., J. Biol. Chem. 262:13979 (1987); Ho et al., J. Biol. Chem. 262:13973 (1987); and U.S. Pat. No. 4,957,735 to Huang et al., each of which is incorporated herein by reference in its entirety).

In some embodiments, the inhibitor of CXCL12 signaling and the immune checkpoint inhibitor are administered by the same route. In other embodiments, the inhibitors are administered by different routes, e.g., by the route most suitable for each agent. For example, the immune checkpoint inhibitor may be administered systemically (e.g., intravenously) and the inhibitor of CXCL12 signaling may be administered locally (e.g., directly into a tumor or into a body cavity containing the tumor) or the inhibitor of CXCL12 signaling may be administered systemically and the immune checkpoint inhibitor may be administered locally. In some embodiments, the immune checkpoint inhibitor may be administered by intravenous infusion and the inhibitor of CXCL12 signaling may be administered by subcutaneous injection or by subcutaneous pump to a local tumor site or for systemic delivery.

As a further aspect, the invention provides pharmaceutical formulations and methods of administering the same to achieve any of the therapeutic effects (e.g., cancer treatment) discussed above. The pharmaceutical formulation may comprise any of the reagents discussed above in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The agents of the invention can be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the agent (including the physiologically acceptable salts thereof) is typically admixed with, inter alia, an acceptable carrier. The carrier can be a solid or a liquid, or both, and is preferably formulated with the agent as a unit-dose formulation, for example, a tablet, which can contain from 0.01 or 0.5% to 95% or 99% by weight of the agent. One or more agents can be incorporated in the formulations of the invention, which can be prepared by any of the well-known techniques of pharmacy.

A further aspect of the invention is a method of treating subjects in vivo, comprising administering to a subject a pharmaceutical composition comprising the inhibitors of the invention in a pharmaceutically acceptable carrier, wherein the pharmaceutical composition is administered in a therapeutically effective amount. Administration of the inhibitors of the present invention to a human subject or an animal in need thereof can be by any means known in the art for administering compounds.

The formulations of the invention include those suitable for oral, rectal, perianal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal, and inhalation administration, administration to the liver by intraportal delivery, as well as direct organ injection (e.g., into the liver, into the brain for delivery to the central nervous system, into the pancreas, or into a tumor or the tissue surrounding a tumor). The most suitable route in any given case will depend on the nature and severity of the condition being treated and on the nature of the particular agent which is being used.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the inhibitors can be administered in solid dosage forms, such as capsules, tablets, and powders, or in liquid dosage forms, such as elixirs, syrups, and suspensions. Inhibitors can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the inhibitors in a flavored base, usually sucrose and acacia or tragacanth; and pastilles comprising the inhibitors in an inert base such as gelatin and glycerin or sucrose and acacia.

Formulations of the present invention suitable for parenteral administration comprise sterile aqueous and non-aqueous injection solutions of the inhibitors, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising inhibitors of the invention, in a unit dosage form in a sealed container. The inhibitors are provided in the form of a lyophilizate which is capable of being reconstituted with a suitable pharmaceutically acceptable carrier to form a liquid composition suitable for injection thereof into a subject. The unit dosage form typically comprises from about 1 mg to about 10 grams of the inhibitors. When the inhibitors are substantially water-insoluble, a sufficient amount of emulsifying agent which is pharmaceutically acceptable can be employed in sufficient quantity to emulsify the inhibitors in an aqueous carrier. One such useful emulsifying agent is phosphatidyl choline.

Formulations suitable for rectal administration are preferably presented as unit dose suppositories. These can be prepared by admixing the inhibitors with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the polypeptides. Suitable formulations comprise citrate or bis/tris buffer (pH 6) or ethanol/water and contain from 0.1 to 0.2M of the compound.

The inhibitors can alternatively be formulated for nasal administration or otherwise administered to the lungs of a subject by any suitable means, e.g., administered by an aerosol suspension of respirable particles comprising the inhibitors, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer, Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the inhibitors can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the inhibitors can likewise be produced with any solid particulate medicament aerosol generator, by techniques known in the pharmaceutical art.

Alternatively, one can administer the inhibitors in a local rather than systemic manner, for example, in a depot or sustained-release formulation.

Further, the present invention provides liposomal formulations of the inhibitors disclosed herein and salts thereof The technology for forming liposomal suspensions is well known in the art. When the inhibitors or salt thereof is an aqueous-soluble salt, using conventional liposome technology, the same can be incorporated into lipid vesicles. In such an instance, due to the water solubility of the inhibitors or salt, the inhibitors or salt will be substantially entrained within the hydrophilic center or core of the liposomes. The lipid layer employed can be of any conventional composition and can either contain cholesterol or can be cholesterol-free. When the inhibitors or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the inhibitors disclosed herein or salts thereof, can be lyophilized to produce a lyophilizate which can be reconstituted with a pharmaceutically acceptable carrier, such as water, to regenerate a liposomal suspension.

In the case of water-insoluble inhibitors, a pharmaceutical composition can be prepared containing the water-insoluble inhibitors, such as for example, in an aqueous base emulsion. In such an instance, the composition will contain a sufficient amount of pharmaceutically acceptable emulsifying agent to emulsify the desired amount of the inhibitors. Particularly useful emulsifying agents include phosphatidyl cholines and lecithin.

In particular embodiments, the inhibitors are administered to the subject in a therapeutically effective amount, as that term is defined above. Dosages of pharmaceutically active inhibitors can be determined by methods known in the art, see, e.g., Remington's Pharmaceutical Sciences (Maack Publishing Co., Easton, Pa). The therapeutically effective dosage of any specific inhibitor will vary somewhat from inhibitor to inhibitor, and patient to patient, and will depend upon the condition of the patient and the route of delivery. As a general proposition, a dosage from about 0.1 to about 50 mg/kg will have therapeutic efficacy, with all weights being calculated based upon the weight of the inhibitors, including the cases where a salt is employed. Toxicity concerns at the higher level can restrict intravenous dosages to a lower level such as up to about 10 mg/kg, with all weights being calculated based upon the weight of the inhibitor, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly to about 22 μmol/kg and to 33 μmol/kg of the inhibitors for intravenous or oral administration, respectively.

In particular embodiments of the invention, more than one administration (e.g., two, three, four, or more administrations) can be employed over a variety of time intervals (e.g., hourly, daily, weekly, monthly, etc.) to achieve therapeutic effects.

Other delivery systems can include time-release, delayed release or sustained release delivery systems. Such systems can avoid repeated administrations of the inhibitors, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075, 109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like.

In one embodiment, the inhibitors are administered in a time-release, delayed release or sustained release delivery system. In one embodiment, the time-release, delayed release or sustained release delivery system comprising the inhibitors is inserted directly into the tumor. In one embodiment, the time-release, delayed release or sustained release delivery system comprising the inhibitors is implanted in the patient proximal to the tumor. Additional implantable formulations are described, for example, in U.S. Patent App. Pub. No. 2008/0300165, which is incorporated herein by reference in its entirety.

In addition, important embodiments of the invention include pump-based hardware delivery systems, some of which are adapted for implantation. Such implantable pumps include controlled-release microchips. A preferred controlled-release microchip is described in Santini, J T Jr. et al., Nature, 1999, 397:335-338, the contents of which are expressly incorporated herein by reference.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The present invention finds use in veterinary and medical applications. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults.

Another aspect of the invention relates to a composition comprising an inhibitor of CXCL12 signaling and an immune checkpoint inhibitor.

A further aspect of the invention relates to a kit of parts comprising a first container comprising an inhibitor of CXCL12 signaling and a second container comprising an immune checkpoint inhibitor.

The inhibitor of CXCL12 signaling and the immune checkpoint inhibitor in the composition or the kit of parts may be any of the agents described above.

The container may be, without limitation, a vial containing a single dose or multiple doses of the inhibitor or a prefilled syringe containing the inhibitor.

In one embodiment, the composition or kit of parts further comprises instructions in a readable medium for dosing and/or administration of the inhibitor of CXCL12 signaling and the immune checkpoint inhibitor. The term “readable medium” as used herein refers to a representation of data that can be read, for example, by a human or by a machine. Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (ROM); random access memory (RAM); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (QR Code) or other matrix barcode.

The present invention is more particularly described in the following examples that are intended as illustrative only since numerous modifications and variations therein will be apparent to those skilled in the art.

### EXAMPLE 1

### Treatment of Ovarian Cancer

Levels of intratumoral FoxP3⁺ regulatory T cells (T_{reg}) can be reduced by AMD3100 (plerixafor), an antagonist of the CXCL12 receptor CXCR4 (Righi et al., Cancer Res. 71:5522 (2011)) Inhibitors of the immune checkpoint proteins PD-1 or PD-L1 can increase anti-tumor effector T cell responses (Curran et al., Proc. Natl Acad. Sci. 107:4275 (2010)), while antagonism of CXCR4 can decrease T_{reg} infiltration, thereby increasing antitumor effects. The objective of this study was to test in a murine model for ovarian cancer a combination immune therapy that involves the checkpoint blockade of PD-1/PD-L1 pathway to restore function of tumor antigen-specific cytotoxic T cells and CXCL12/CXCR4 blockade by the CXCR4 antagonist AMD3100 to selectively reduce intratumoral regulatory T cells (T_{reg}s).

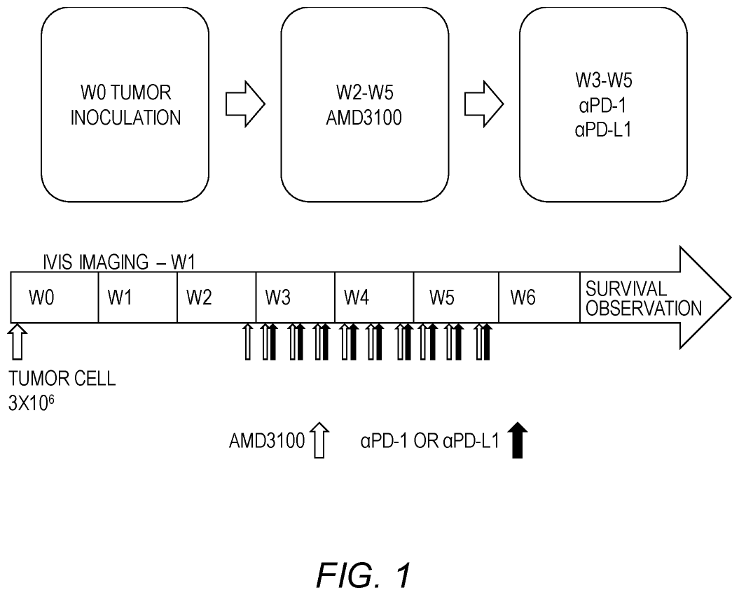

Luciferase-expressing ID-8 ovarian cancer cells (3×10⁶ cells) were injected into C57BL/6 female mice (30/group). Tumor initiation was confirmed 20 days later by IVIS imaging, and tumor-bearing animals were randomized into the treatment groups indicated in Table 2. Starting three weeks post tumor inoculation, AMD3100 (3 mg/kg) was injected IP every other day for three weeks alternating with αPD-1 or αPD-L1 (200 μg/mouse) by IP injection. The protocol is shown in FIG. 1. 3 mg/kg AMD3100 is the murine equivalent of the approved human dose of 0.24 mg/kg based on allometric scaling.

TABLE 2

| Group | AMD3100 | Anti-PD-1 | Anti-PD-L1 |
|---|---|---|---|
| 1 | 0 | 0 | 0 |
| 2 | 3 mg/kg/dose | 0 | 0 |
| 3 | 0 | 200 μg/mouse/dose | 0 |
| 4 | 0 | 0 | 200 μg/mouse/dose |
| 5 | 3 mg/kg/dose | 200 μg/mouse/dose | 0 |
| 6 | 3 mg/kg/dose | 0 | 200 μg/mouse/dose |

Some animals from each treatment group were euthanized by i.p. administration of Ketamine (9 mg/ml in saline) and Xylazine (0.9 mg/ml in saline) a week after the last treatment for evaluation of tumor-infiltrating lymphocytes. Tumors were harvested and processed for immune profiling. The tumor-infiltrated lymphocytes were labeled and evaluated by flow cytometry (antibody panels are shown in Table 3). The remaining mice in each group were observed for survival.

TABLE 3

| Marker | Label |
|---|---|
| CD3 | Bv421 |
| CD4 | BV711 |
| CD8 | PE/CY7 |
| CD25 | APC |
| CD11b | BV605 |
| Gr-1 | FITC |
| CD44 | AF-700 |
| CD27 | BV510 |
| CD40L | PerCp |
| PD-1 | BV785 |
| FoxP3 | PE |
| IFNr | APC/CY7 |
| IL2 | PE594 |
| Zombie Live/Dead | DAPI |

### Methods

Tissue Culture: The ID8 ovarian cancer cells, a kind gift from Kathy Roby (University of Kansas Medical Center, Kansas City, Kans.), were transfected with luciferase lentiviral vector and a clone stably expressing luciferase was recovered, here named Luc-ID8. Cells were maintained at 37° C. in DMEM with 2 mmol/L L-glutamine, 10 units/ml penicillin, 10 μg/ml streptomycin, and 10% fetal bovine serum in a humidified atmosphere with 5% CO₂. Cells were cultured until 80% confluent, then harvested with Trypsin EDTA (Mediatech) for animal injections.

Treatment Agents: AMD3100 was purchased from Abcam. 10 mg power was dissolved in 16.7 ml saline to achieve concentration of 0.6 mg/ml and 100 μl was injected for each treatment. Anti-mouse PD-1 and anti-mouse PD-L1 were purchased from Biocell. The original antibody was diluted in saline to the concentration of 2 mg/ml. 100 μl (200 μg) per injection was administered to each mouse.

Animals: Five-week-old female C57BL/6 mice were obtained from Jackson Laboratory and maintained in a gnotobiotic animal facility.

Animal Housing: The study was performed in animal rooms provided with filtered air at a temperature of 70° F.±5° F. and 50%±20% relative humidity. Animal rooms were set to maintain a minimum of 12 to 15 air changes per hour. The room was on an automatic timer for a light/dark cycle of 12 hours on and 12 hours off with no twilight. Sterilized Bed-O-Cobs® bedding was used. Bedding was changed a minimum of once per week. Cages, tops, bottles, etc. were washed with a commercial detergent and allowed to air dry. Prior to use, these items were wrapped and autoclaved. A commercial disinfectant was used to disinfect surfaces and materials introduced into the hood. Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number and treatment group marked all cages. The temperature and relative humidity were recorded during the study, and the records retained.

Animal Diet: Animals were fed with sterile Labdiet® 5053 (pre-sterilized) rodent chow and sterile water was provided ad libitum.

Animal Randomization and Allocations: Mice were randomly and prospectively divided into different treatment groups prior to the initiation of treatment. Each animal was identified by ear punching corresponding to an individual number. A cage card was used to identify each cage and marked with the study number (CAN-01), treatment group number and animal numbers.

Tumor Establishment: After one week resting following receipt into the animal facility, 3×10⁶ LUC-ID8 cells were administrated i.p. per mouse. The IVIS Spectrum animal imaging system (PerkinElmer) was utilized to evaluate the establishment of tumor on day 20 after tumor cell injection. Mice were injected intraperitoneally with 150 mg/kg body weight of D-luciferin (Regis Technologies) 5 min in advance of imaging by IVIS Spectrum.

Mouse survival: For survival studies, the mice were observed daily beginning at the end of treatment, and the life span of each mouse recorded.

Flow cytometry: Tumors were mechanically disaggregated using razor blades, and cells were further separated by incubation at 37° C. for 2 hours in RPMI 1640 with collagenase type IV (2 mg/ml, Sigma), hyaluronidase (0.1 mg/ml, Sigma), and BSA (2 mg/ml, Sigma). Cell suspensions were passed through 100 μm filters to remove any remaining aggregates. Cells were washed with staining buffer (Biolegend) and stained with conjugated antibodies. All live cells were determined by the LIVE/DEAD® staining (ThermoFisher). Intracellular protein detection was realized by fixation/permeabilization reagents from BioLegend or eBioscience. Flow cytometry were performed using BD LSRFortessa X-20 (BD Biosciences) and data were analyzed by FlowJo V10.

Assessment of Results: Prism 6.0 software (GraphPad Software) was used for all statistical analyses. Statistical differences between three or more experimental groups were analyzed using Two-Way ANOVA, followed by Tukey's multiple comparison tests or Student T tests. Survival was analyzed with the Log-rank test.

### Results and Discussion

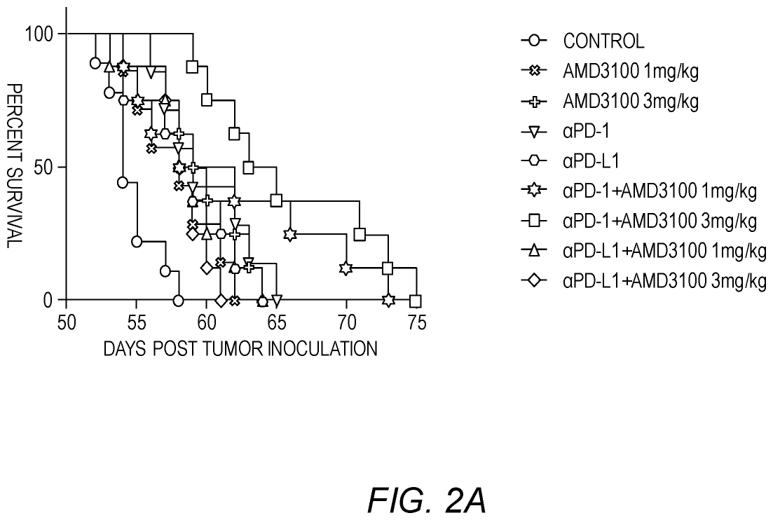

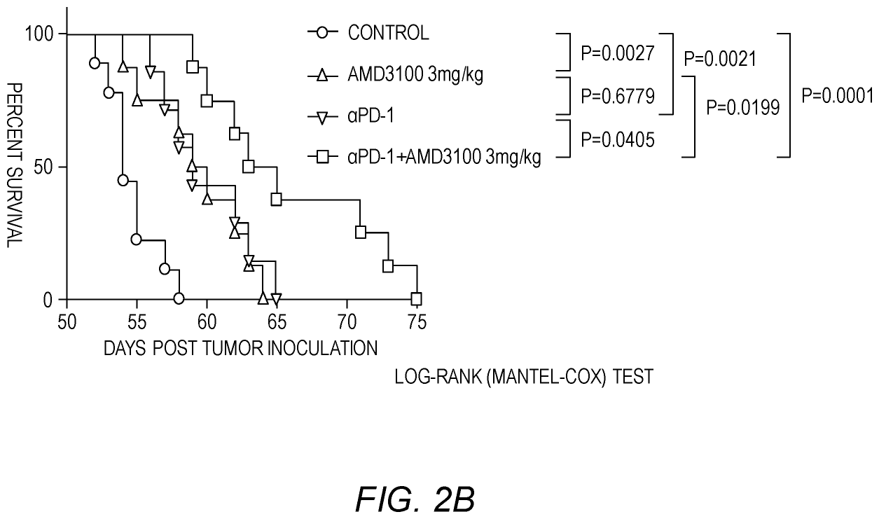

Combination therapy significantly increased the survival time of ID8 tumor bearing mice. Survival of tumor-bearing animals was extended in all treatment groups relative to the saline treated control group (FIGS. 2A-2B). The two groups receiving AMD3100+αPD-1 combination treatment exhibited the greatest extension of survival times.

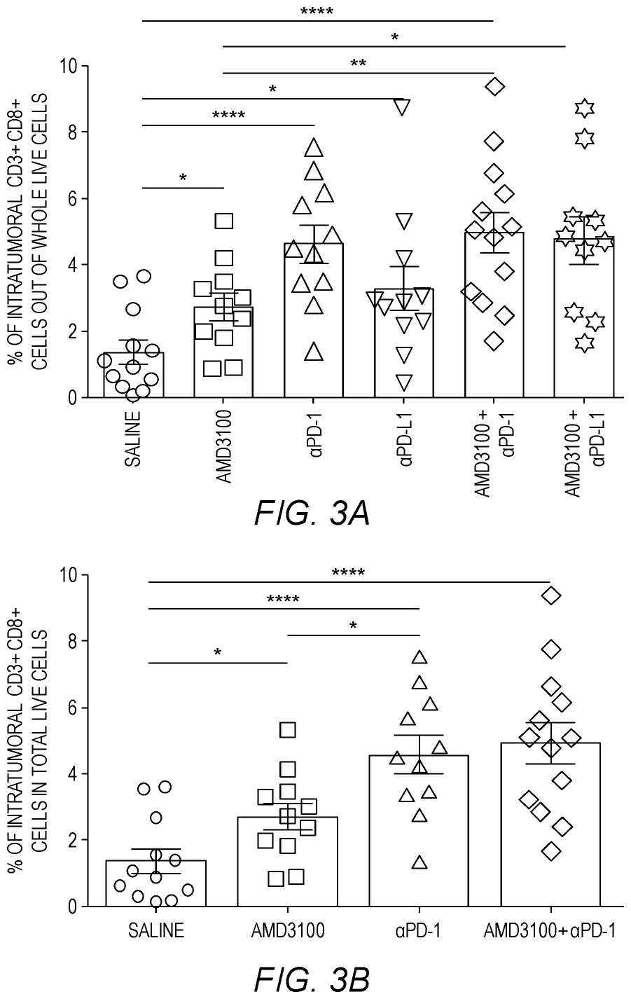

Monotherapy and combination therapy enhanced intratumoral T cell infiltration. αPD-1 or αPD-L1 alone or in combination with AMD3100 significantly increased the proportions of CD8+ T cells in tumors compared to saline control treatment (FIGS. 3A-3B).

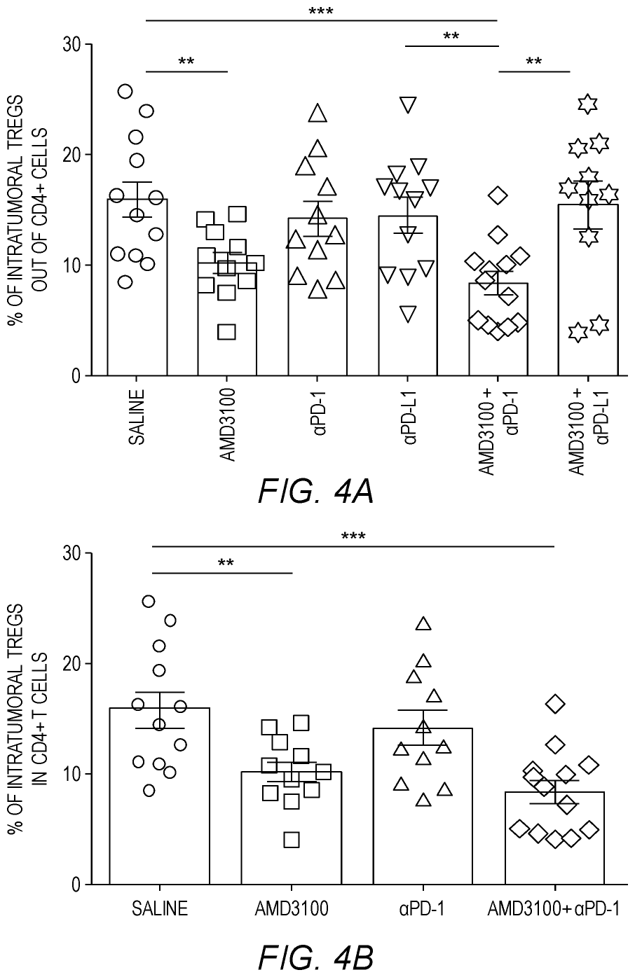

AMD3100 inhibited intratumor Tregs. The proportions of intratumor Tregs were significantly reduced in tumor-bearing mice treated by AMD3100 alone and in combination with αPD-1 compared to saline control treatment (FIGS. 4A-4B).

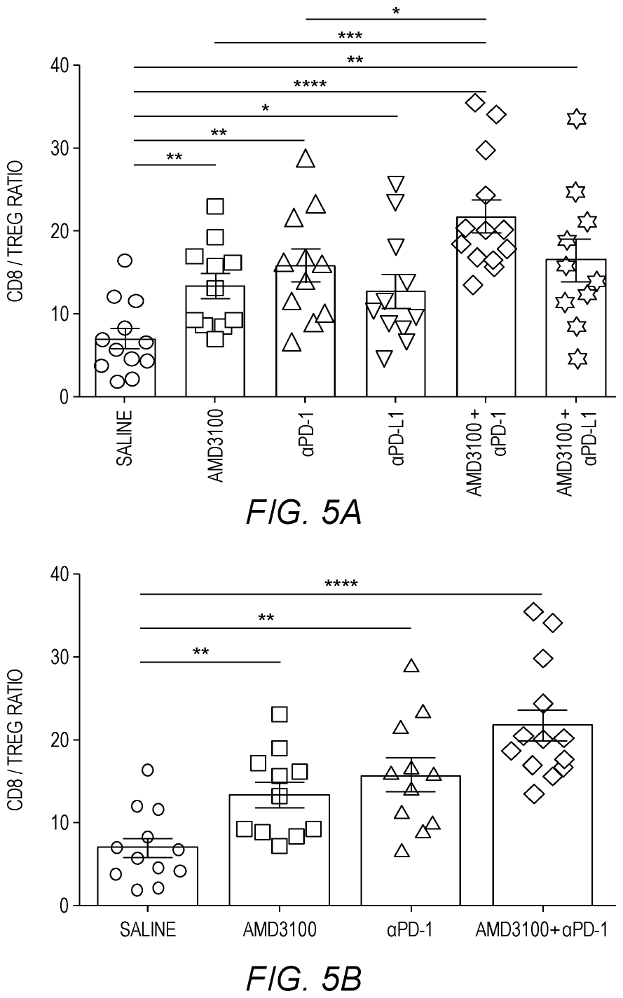

Monotherapy and combination therapy increased ratios of CD8+ T cells to Tregs in tumors. αPD-1 or αPD-L1 alone or in combination with AMD3100 significantly increased ratios of CD8+ T cells to Tregs in tumors compared to saline control treatment (FIGS. 5A-5B).

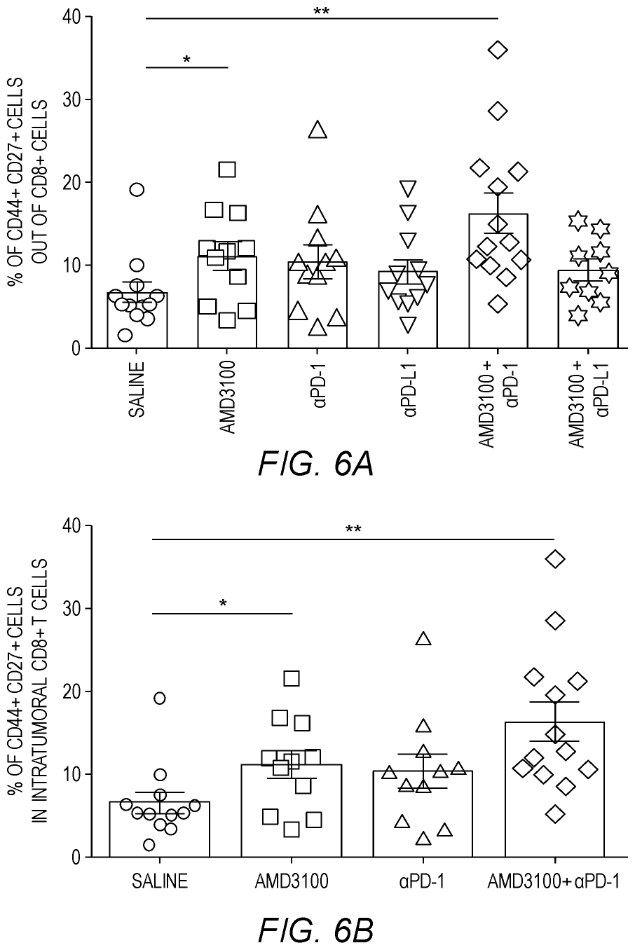

AMD3100 alone and in combination with αPD-1 increased memory T cells in tumors. AMD3100 alone and in combination with αPD-1 significantly increased intratumor CD8+ memory T cells indicated by CD27+CD44+ population in TILs (FIGS. 6A-6B).

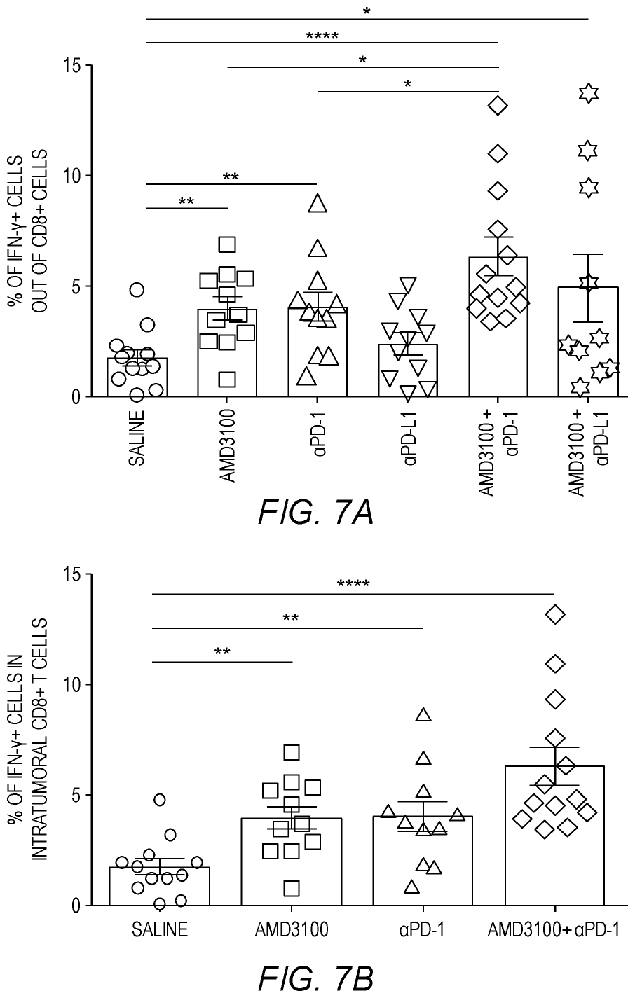

AMD3100 or αPD-1 alone and their combination enhanced intratumor T cell effector function. IFN-γ expression was significantly increased in intratumor CD8+ T cells in AMD3100 or αPD-1 monotherapy and their combination therapy compared to saline control treatment (FIGS. 7A-7B).

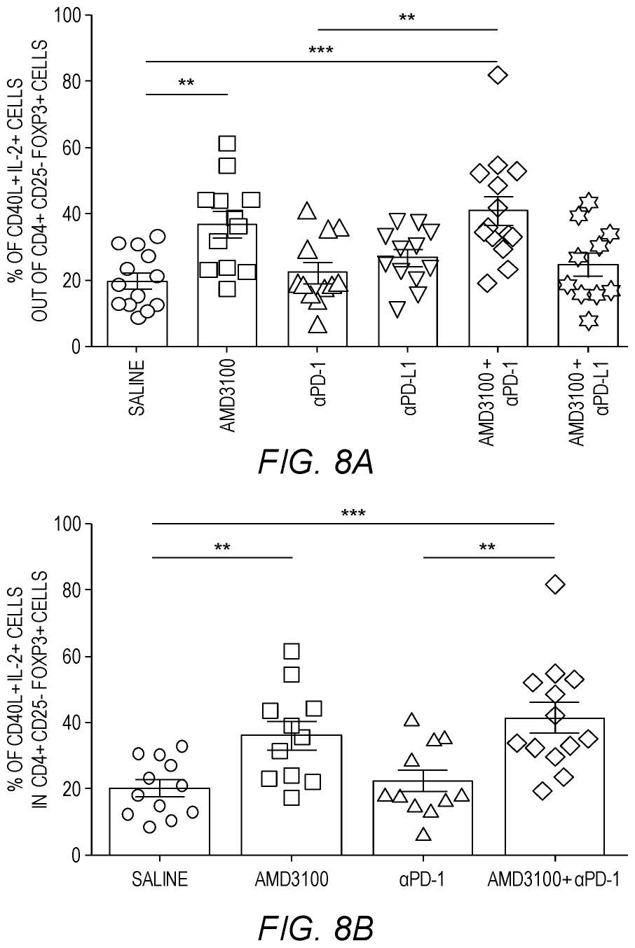

AMD3100 resulted in replacement of Tregs by helper T cells in TILs. The proportions of CD40L+IL-2+ cells in CD4+FoxP3+CD25− population, referred to helper-like T cells were significantly increased in tumors treated by AMD3100 alone and in combination with αPD-1 but not by αPD-1 alone (FIGS. 8A-8B).

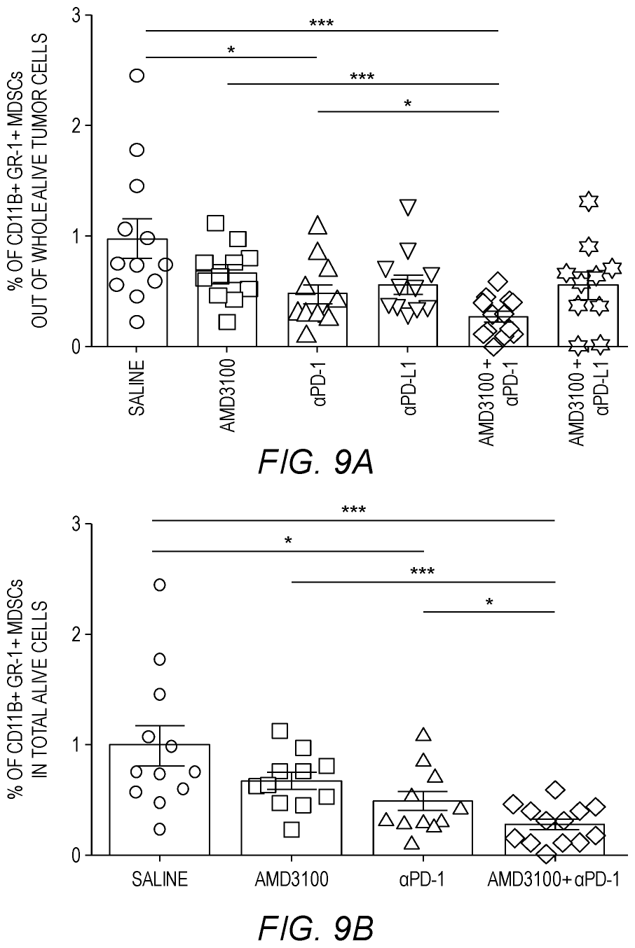

αPD-1 alone and in combination with AMD3100 significantly decreased intratumor MDSCs. Myeloid derived suppressive cells (MDSCs) indicated by CD11b+ and Gr-1+ were significantly decreased by αPD-1 alone and further decreased by its combination with AMD3100 (FIGS. 9A-9B).

In conclusion, anti-PD-1 or AMD3100 significantly extended the survival time of ID8 tumor bearing mice when given as monotherapy compared to saline control treatment. Combination of AMD3100 and anti-PD-1 increased the survival compared to single agent administration. αPD-1 or αPD-L1 alone or in combination with AMD3100 enhanced intratumor infiltration of CD8+ T cells. AMD3100 or αPD-1 alone and their combination enhanced IFN-γ of intratumor CD8+ T cells. AMD3100 alone and in combination with αPD-1 decreased intratumor Tregs. αPD-1 or αPD-L1 alone or in combination with AMD3100 increased ratios of CD8+ T cells to Tregs in tumors. AMD3100 alone and in combination with αPD-1 increased memory T cells in tumors. AMD3100 promoted replacement of Tregs by helper T cells in tumors. αPD-1 alone and in combination with AMD3100 decreased intratumor myeloid derived suppressor cells.

The foregoing is illustrative of the present invention, and is not to be construed as limiting thereof. The invention is defined by the following claims, with equivalents of the claims to be included therein.

1. A method of treating cancer in a subject in need thereof, comprising delivering to the subject an inhibitor of CXCL12 signaling and an immune checkpoint inhibitor, thereby treating the cancer.

2. A method for enhancing an immune response against a cancer in a subject in need thereof, comprising delivering to the subject an inhibitor of CXCL12 signaling and an immune checkpoint inhibitor, thereby enhancing the immune response against the cancer.

3. The method of claim 1, wherein the inhibitor of CXCL12 signaling is a CXCR4 antagonist.

4. The method of claim 3, wherein the inhibitor of CXCL12 signaling is AMD3 100 (plerixafor).

5. The method of claim 1, wherein the immune checkpoint inhibitor is an inhibitor of PD-1 or PD-L1.

6. The method of claim 1, wherein the immune checkpoint inhibitor is an antibody that specifically binds PD-1 or PD-L1.

7. The method of claim 1, wherein the immune checkpoint inhibitor is nivolumab, pembrolizumab, ipilimumab, durvalumab, or atezolizumab.

8. (canceled)

9. The method of claim 1, wherein the cancer is a solid tumor, and wherein the cancer is ovarian cancer, fallopian tube cancer, cervical cancer, vaginal cancer, rectal cancer, pancreatic cancer, cholangiocarcinoma, peritoneal cancer, mesothelioma, non-small cell lung cancer, kidney cancer, bladder cancer, Hodgkin lymphoma, or head and neck cancer.

10-11. (canceled)

12. The method of claim 1, wherein the cancer is a leukemia.

13. The method of claim 1, wherein the inhibitor of CXCL12 signaling and the immune checkpoint inhibitor are delivered to the subject in the same composition.

14. The method of claim 1, wherein the inhibitor of CXCL12 signaling and the immune checkpoint inhibitor are delivered to the subject in separate compositions.

15. The method of claim 1, wherein the inhibitor of CXCL12 signaling and the immune checkpoint inhibitor are delivered to the subject sequentially.

16. (canceled)

17. The method of claim 1, further comprising delivering to the subject an anti-cancer agent.

18. The method claim 17, wherein the anti-cancer agent is a chemotherapeutic agent, a radiotherapeutic agent, an immunotherapeutic agent, a natural killer cell, a T cell, or an antibody specific for the cancer.

19-23. (canceled)

24. A composition comprising an inhibitor of CXCL12 signaling and an immune checkpoint inhibitor.

25. A kit of parts comprising a first container comprising an inhibitor of CXCL12 signaling and a second container comprising an immune checkpoint inhibitor.

26. The kit of parts of claim 25, wherein the inhibitor of CXCL12 signaling is a CXCR4 antagonist.

27. The kit of parts of claim 26, wherein the inhibitor of CXCL12 signaling is AMD3100 (plerixafor).

28. The kit of parts of claim 25, wherein the immune checkpoint inhibitor is an inhibitor of PD-1 or PD-L1.

29. (canceled)

30. The kit of parts of claim 25, wherein the immune checkpoint inhibitor is nivolumab, pembrolizumab, ipilimumab, durvalumab, or atezolizumab.

31. (canceled)
